(19) United States
(12) Patent Application Publication
(10) Pub. No.: US 2017/0057197 A1
(43) Pub. Date: Mar. 2, 2017

(54) METHOD, KIT, AND TAPE FOR ATTACHING OBJECTS TO A SUBJECT

(71) Applicant: SPIDERTECH INC., Toronto (CA)

(72) Inventor: Ray Arbesman, Toronto (CA)

(73) Assignee: SPIDERTECH INC., Toronto (CA)

(21) Appl. No.: 15/244,078

(22) Filed: Aug. 23, 2016

Related U.S. Application Data
(60) Provisional application No. 62/212,635, filed on Sep. 1, 2015.

(51) Int. Cl. B32B 3/16; A63B 71/12; B32B 5/02; B32B 7/06; B32B 7/14; A61F 13/02; A41D 13/00

(52) U.S. Cl. CPC B32B 3/16; A61F 13/0269; A63B 71/12; A41D 13/0002; B32B 7/06; B32B 7/14; B32B 5/024

(57) ABSTRACT

A tape for attaching objects to a subject includes a strip of high stretch woven material having a first face and an opposed second face. A first adhesive is on the first face in a discontinuous pattern. A second adhesive is on the second face.

### CROSS REFERENCES TO RELATED APPLICATIONS

This application claims the benefit of U.S. Provisional Patent application No. 62/212,635, filed on Sep. 1, 2015, which is incorporated herein by reference in its entirety.

### FIELD

The specification relates to methods, kits, and tapes for attaching objects to a subject. More particularly, the specification relates to methods, kits, and tapes for adhesively attaching objects such as body protective gear to a subject's skin.

### BACKGROUND

U.S. Pat. No. 5,861,348 (Kase) purports to disclose a body-adhesive tape having adhesive properties preventing the tape from peeling off due to sweat when it is applied in an overlapped manner, and which also has good moisture permeability. In the body-adhesive tape, a base cloth is formed from a stretchable warp thread and a standard weft thread. On one side of the base cloth a plurality of pressure-sensitive adhesive layers are formed separated by appropriate gaps. The pressure-sensitive adhesive layers are formed so that they meander left and right along the length of the tape. At the gaps, the base cloth is exposed so there is permeability. Since the gap meanders, it is longer than the tape, and thus there is more exposure of the weave and more permeability. The pressure-sensitive adhesive layers meander so that the tape can be attached anywhere on the body and can respond to tension due to body movement from any direction. The base cloth is processed for water repellency so that the tapes can be attached in an overlapping manner and no sweat or the like will be absorbed, and the upper tape will not tend to peel off.

### SUMMARY

The following summary is intended to introduce the reader to various aspects of the applicant's teaching, but not to define or delimit any invention.

According to some aspects, a tape for attaching objects to a subject is disclosed. The tape comprises a strip of high stretch woven material having a first face and an opposed second face. A first adhesive is on the first face in a discontinuous pattern. A second adhesive is on the second face.

The second adhesive may be on the second face in a discontinuous pattern.

The first adhesive and the second adhesive may overlay each other.

The first face may include a first set of spaced apart adhesive-covered portions that are covered by the adhesive, and a first set of spaced apart adhesive-free portions that are free of the adhesive. The second face may include a second set of spaced apart adhesive-covered portions that are covered by the adhesive, and a second set of spaced apart adhesive-free portions that are free of the adhesive.

The first adhesive and the second adhesive may be a skin-safe adhesive. The first adhesive and the second adhesive may be a poly-acrylic adhesive.

The woven material may be woven cotton.

The strip may comprise an anchor portion and a plurality of fingers extending away from the anchor portion.

The strip may have an unstretched length, and may be elastically stretchable to a stretched length that is at least 1.4 times the unstretched length.

According to some aspects, a kit for attaching objects to a subject is disclosed. The kit comprises a tape comprising a strip of high stretch woven material having a first face and an opposed second face, a first adhesive on the first face in a discontinuous pattern, and a second adhesive on the second face. A first release liner is on the first face. A second release liner is on the second face.

The tape, the first release liner, and the second release liner may be provided in a roll.

The tape, the first release liner, and the second release liner may be provided in individual precut pieces. The tape of each individual precut piece may comprise an anchor portion and a plurality of fingers extending away from the anchor portion.

The kit may further comprise instructions directing a user to remove the first release liner and adhere the first face to a subject's skin, and remove the second release liner and adhere an object to the second face.

The second adhesive may be on the second face in a discontinuous pattern.

The first face may include a first set of spaced apart adhesive-covered portions that are covered by the adhesive, and a first set of spaced apart adhesive-free portions that are free of the adhesive. The second face may include a second set of spaced apart adhesive-covered portions that are covered by the adhesive, and a second set of spaced apart adhesive-free portions that are free of the adhesive.

The first adhesive and the second adhesive may be a skin-safe adhesive. The first adhesive and the second adhesive may be a poly-acrylic adhesive.

The woven material may be woven cotton.

The strip may comprise an anchor portion and a plurality of fingers extending away from the anchor portion.

The strip may have an unstretched length, and may be elastically stretchable to a stretched length that is at least 1.4 times the unstretched length.

According to another aspect, a method for attaching objects to a subject is disclosed. The method comprises a) removing a first release liner from a first face of a strip of high stretch woven material; b) adhering the first face to the subject's skin via a first adhesive, the first adhesive in a discontinuous pattern on the first face; c) removing a second release liner from a second face of the strip; and d) adhering the object to the subject via a second adhesive on the second face.

Step b) may comprise adhering the first face to the subject's skin while the strip is in an unstretched configuration.

The object may be body protective gear or athletic gear.

The second adhesive may be on the second face in a discontinuous pattern.

### BRIEF DESCRIPTION OF THE DRAWINGS

The drawings included herewith are for illustrating various examples of articles, methods, and apparatuses of the present specification and are not intended to limit the scope of what is taught in any way. In the drawings:

FIG. 1 is a top view of an example tape for attaching objects to a subject;

FIG. 2 is a bottom view of the tape of FIG. 1;

FIG. 3 is an enlarged end view of the tape of FIG. 1;

FIG. 3A is an enlarged end view of an alternative tape;

FIG. 4A is a top view of a kit for attaching objects to a subject, including the tape of FIG. 1 (shown in dotted line) between a pair of release liners;

FIG. 4B is a side view of the kit of FIG. 4A;

FIG. 5 is a perspective view of another kit for attaching objects to a subject, including a tape (shown in dotted line) and a pair of release liners provided on a roll;

FIG. 6 is a top view of another kit for attaching objects to a subject, including an individual precut piece of tape (shown in dotted line) between a pair of release liners, wherein the dashed lines represent perforations in the release liners;

FIG. 7 is a perspective view of another kit for attaching objects to a subject, including a set of precut pieces of tape (shown in dotted line) and a pair of release liners provided on a roll, wherein the dashed lines represent perforations in the release liners;

FIG. 8A is a top view showing a first release liner being removed from the tape of FIG. 4;

FIG. 8B is a top view showing the tape of FIG. 8A with the first release liner fully removed therefrom;

FIG. 9 is a schematic front view of a shin, with the first face of the tape (shown in dotted line) of FIG. 8B adhered to the skin;

FIG. 10 is a schematic front view of the shin and tape of FIG. 9, with the second release liner being removed from the tape;

FIG. 11 is a schematic front view of the shin and tape of FIG. 10, with the second release liner fully removed from the tape; and

FIG. 12 is a schematic front view of the shin and tape (shown in dotted line) of FIG. 11, with a shin pad adhered to the second face of the tape.

### DETAILED DESCRIPTION

Various apparatuses or processes will be described below to provide an example of an embodiment of the claimed subject matter. No embodiment described below limits any claim and any claim may cover processes or apparatuses that differ from those described below. The claims are not limited to apparatuses or processes having all of the features of any one apparatus or process described below or to features common to multiple or all of the apparatuses described below. It is possible that an apparatus or process described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

Disclosed herein is a tape for attaching objects to a subject, and related kits and methods. For example, the tape may be used to adhesively attach body-protective gear to a subject's skin. Such body-protective gear may include athletic gear such as but not limited to shin pads, elbow pads, chest pads, knee pads, shoulder pads, thigh pads, neck guards, face guards, and/or goggles. The tape may be used alone to attach the body protective gear to the subject's skin. Alternatively, the tape may be used together with other attachment mechanisms. For example, a shin pad may be secured to a subject's leg by a strap, and the tape may be used to hold the shin pad in place and prevent or inhibit movement of the shin pad (e.g. rotation around the leg or movement up or down the leg) while in use. For further example, the tape may be used to adhesively attach medical equipment to a subject's skin. Such medical equipment may include but is not limited to orthopedic braces, electronic sensors, tubing, and/or catheters.

The tape may in some examples provide kinesiologic benefits to the subject, in addition to attaching the object to the subject. For example, the tape may be suitable for use as kinesiology tape, in addition to being suitable for attaching objects to a subject.

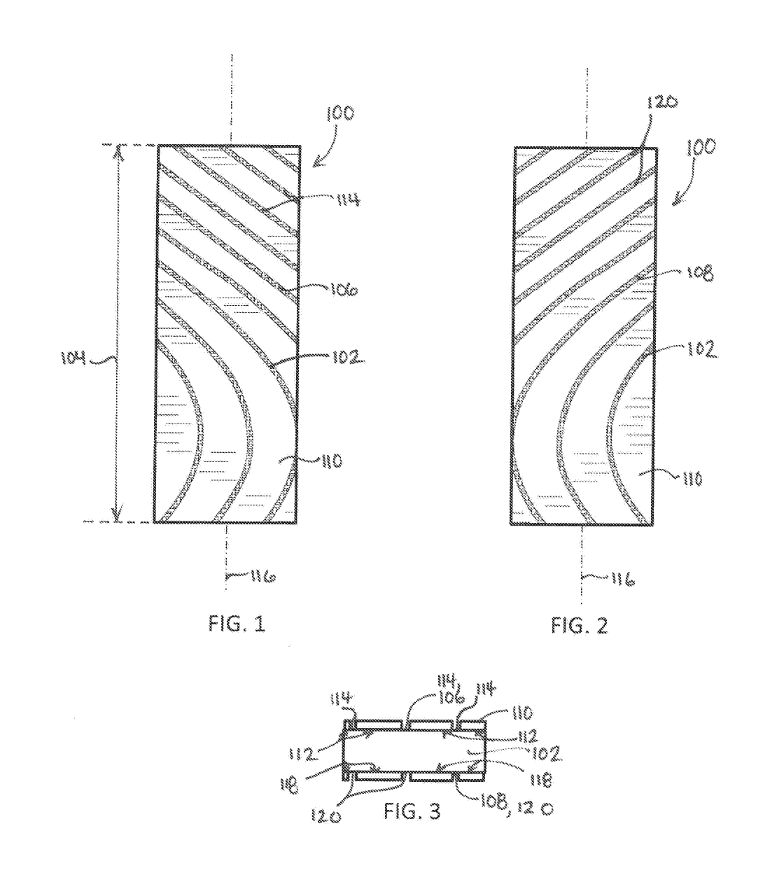

Referring now to FIGS. 1 to 3, an example tape 100 is shown. In the example shown, the tape 100 includes a strip 102 of high stretch woven material. As used herein, the term ‘high stretch’ indicates that the strip 102 is highly stretchable in at least one direction. For example, the strip may be highly stretchable along its length. For example, the woven material may be a woven cotton having a stretch ratio of at least 1.4 along its length (i.e. the strip 102 may have an unstretched length 104, and may be elastically stretchable to a length that is at least 1.4 times the unstretched length 104). The strip 102 may additionally be highly stretchable along its width, or may be minimally-stretchable or non-stretchable along its width. The term ‘minimally’ stretchable indicates that the strip 102 is stretchable along its width, but less stretchable than it is along its length. The term ‘non-stretchable’ indicates that the strip 102 is not stretchable along its width, or is stretchable to only a negligible extent along its width. In some particular examples, the high stretch woven material may be similar to or the same as the base material used in kinesiology tapes, which is highly stretchable along its length and minimally-stretchable along its width.

The use of a woven material may allow for breathability of the tape 100. Furthermore, the use of a high stretch material may allow for the tape 100 to accommodate movement of the subject as well as movement of the object.

The strip 102 has a first face 106 (shown in FIG. 1) that in use faces the skin of the subject, and an opposed second face 108 (shown in FIG. 2) that in use faces an object that is adhered to the skin.

An adhesive 110 is on both the first face 106 and the second face 108. The adhesive 110 on the first face 106 may also be referred to herein as a “first adhesive”, and the adhesive 110 on the second face 108 may also be referred to herein as a “second adhesive”.

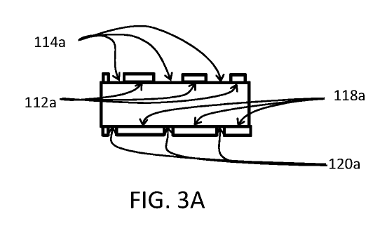

Referring to FIGS. 1 and 3, in the example shown, the adhesive 110 is on the first face 106 in a discontinuous pattern. That is, the first face 106 includes a set of adhesive-covered portions 112 (i.e. portions that are covered by the adhesive 110), and a set of adhesive-free portions 114 (i.e. portions that are free of adhesive 110, so that the first face is exposed). The adhesive-covered portions 112 and adhesive-free portions 114 are provided in an alternating pattern, so that the adhesive-covered portions 112 are spaced apart from each other, and the adhesive-free portions 114 are spaced apart from each other.

Referring still to FIG. 1, the tape 100 extends along a longitudinal axis 116. At least some of the adhesive-free portions 114 may extend non-parallel to the longitudinal axis 116. Furthermore, at least some of the adhesive-free portions 114 may extend non-perpendicular to the longitudinal axis 116. For example, the adhesive-free portions 114 may be curved, and may extend generally diagonally with respect to the longitudinal axis 116. This may allow for the tape 100 to stretch and accommodate movement in both a lengthwise direction and a widthwise direction (as well as additional directions that have both a lengthwise and widthwise component), even when adhered to skin. In the example shown, the adhesive-covered portions 112 and adhesive-free portions 114 are provided as curved bands that are arranged in an alternating pattern and extend generally diagonally with respect to the longitudinal axis 116.

Referring to FIGS. 2 and 3, in the example shown, the adhesive 110 is on the second face 108 in a discontinuous pattern, similar to the adhesive 110 on the first face 106. The second face 108 includes a set of adhesive-covered portions 118 (i.e. portions that are covered by the adhesive 110), and a set of adhesive-free portions 120 (i.e. portions that are free of adhesive 110, so that the second face 108 is exposed). The adhesive-covered portions 118 and adhesive-free portions 120 are provided in an alternating pattern, so that the adhesive-covered portions 118 are spaced apart from each other, and the adhesive-free portions 120 are spaced apart from each other.

Similarly to the adhesive 110 on the first face 106, at least some of the adhesive-free portions 120 may extend non-parallel to the longitudinal axis 116. Furthermore, at least some of the adhesive-free portions 120 may extend non-perpendicular to the longitudinal axis 116. For example, the adhesive-free portions 120 may be curved, and may extend generally diagonally with respect to the longitudinal axis 116. This may allow for the tape 100 to stretch and accommodate movement in both a lengthwise direction and a widthwise direction (as well as additional directions that have both a lengthwise and widthwise component), even when adhered to an object. In the example shown, the adhesive-covered portions 118 and adhesive-free portions 120 are provided as curved bands that are arranged in an alternating pattern and extend generally diagonally with respect to the longitudinal axis 116.

Referring to FIG. 3, in the example shown, the adhesive covered portions 112 on the first face 106 and the adhesive covered portions 118 on the second face 108 are aligned with each other. In other words, the adhesive covered portions 112 on the first face 106 and the adhesive covered portions 118 on the second face 108 overlay each other. Furthermore, the adhesive-free portions 114 on the first face and the adhesive-free portions 120 on the second face 108 are aligned with each other (i.e. overlay each other). This provides sections of the tape 100 that are free of adhesive on both faces 106, 108. This may facilitate stretchability of the tape 100, even when in use. For example, when the tape 100 is used to adhere an object to a subject's skin, the adhesive-free portions 114 and 120 will remain non-adhered to the skin and the object, and can stretch to accommodate movement of the subject and the object.

In an alternatives example, the adhesive covered portions on the first face and the adhesive covered portions on the second face may only partially overlie each other. For example, referring to FIG. 3A, the adhesive covered portions 112a on the first face are smaller in width (i.e. narrower) than the adhesive covered portions 118a on the second face (and the adhesive free portions 114a on the first face are larger in width than the adhesive free portions 120a on the second face), and therefore only partially overlay the adhesive covered portions 118a on the second face.

In further alternative examples, the adhesive covered portions 112 on the first face 106 and the adhesive covered portions 118 on the second face 108 may be non-aligned with each other, and the adhesive-free portions 114 on the first face and the adhesive-free portions 120 on the second face 108 may be non-aligned with each other.

The adhesive 110 on the first face 106 and the adhesive 110 on the second face 108 may in some examples be the same adhesive. For example, the adhesive on the first face 106 and the adhesive 110 on the second face 108 may both be a skin-safe adhesive, such as a pressure-activated polyacrylic adhesive used in kinesiology tapes. In alternative examples, the adhesive 110 on the first face 106 and the adhesive 110 on the second face 108 may be different. For example the adhesive 110 on the second face 108 may not be skin safe.

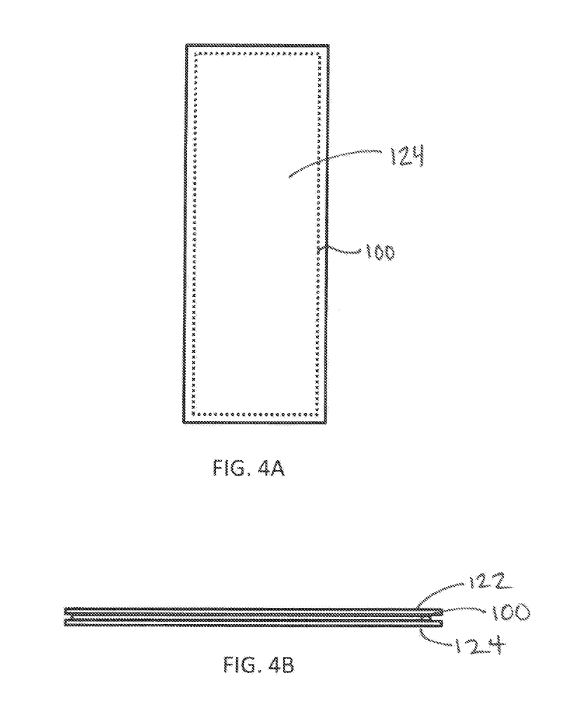

Referring now to FIGS. 4A and 4B, the tape 100 may be provided in a kit with a first release liner 122 on the first face 106 and adhered to the first face 106 by the adhesive 110, and a second release liner 124 (shown in FIG. 4B) on the second face 106 and adhered to the second face 108 by the adhesive 110.

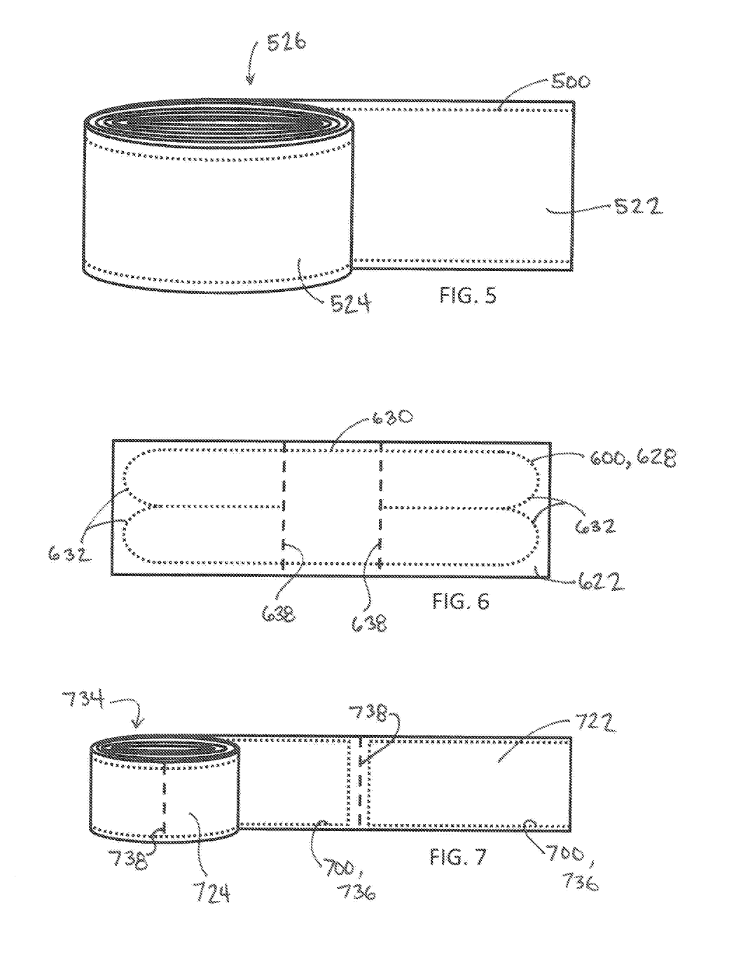

The tape 100 and release liners 122, 124 may be provided in a variety of forms, as shown by way of example in FIGS. 5 to 7. In FIGS. 5 to 7, like features to FIGS. 1 to 4 are shown with like reference numerals, with the first digit incremented to match the Figure number. Referring to FIG. 5, the tape 500 (shown in dotted line), together with the first release liner 522 and second release liner 524, may be provided in a kit on a roll 526. Alternatively, referring to FIG. 6, the tape 600 (shown in dotted line) may be provided as an individual pre-cut piece 628, such as an individual piece cut to an X-shape having an anchor portion 630 and four finger portions 632 extending away from the anchor portion 630. Other non-limiting example shapes include a Y-shape, and an I-shape. The precut piece of tape 628 may be provided in a kit with a first release liner 622 and a second release liner (not shown). Referring to FIG. 7, in yet further alternative examples, the tape 700 and first 722 and second 724 release liners may be provided in a kit of several precut pieces 736 on a roll 734.

In any of the above examples, the first and second release liners may be perforated at various positions to facilitate step-wise removal of the release liners and step-wise application of the tape. For example, referring to FIG. 6, the first 622 and second (not shown) release liners on the X-shaped tape 600 may each include one or more perforated lines 638 extending along a boundary between the anchor portion 630 and the finger portions 632. In use, the first 622 and second release liners may be severed along the perforated line 638, so that the respective portions of the first 622 and second release liners can be removed individually. For further example, referring to FIG. 7, the release liners 730, 732 may include a perforated line 738 between each precut piece 736.

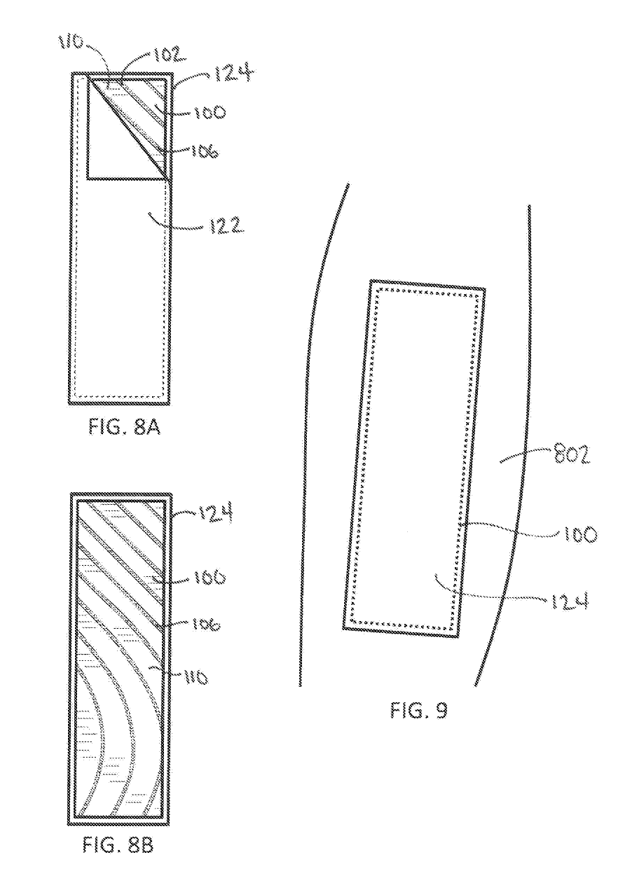

Referring to FIGS. 8 to 11, a method for attaching an object to a subject will be described. More specifically, a method for attaching body-protective gear in the form of a shin pad 800 to a shin 802 will be described. The method will be described with reference to tape 100 and release liners 122 and 124. However, the method may also be carried out with other tapes. Furthermore, the tapes described herein may be used according to other methods.

Referring to FIGS. 8A and 8B, the first release liner 122 may be removed from tape 100 by peeling it from the first face 106 of the strip 102. Referring to FIG. 9, the first face 106 may then be adhered to the subject's skin on the shin 802 via the adhesive 110 on the first face 106. Pressure may then optionally be applied to the tape 100 (e.g. by rubbing the second release liner 124) to activate the adhesive 110. The first face 106 may adhered while the strip 102 is in an unstretched configuration. Alternatively, the first face 106 may be adhered while the strip 102 is in a stretched configuration. Optionally, body hair may be removed from the shin 802 prior to adhering the tape 100 to the shin 802.

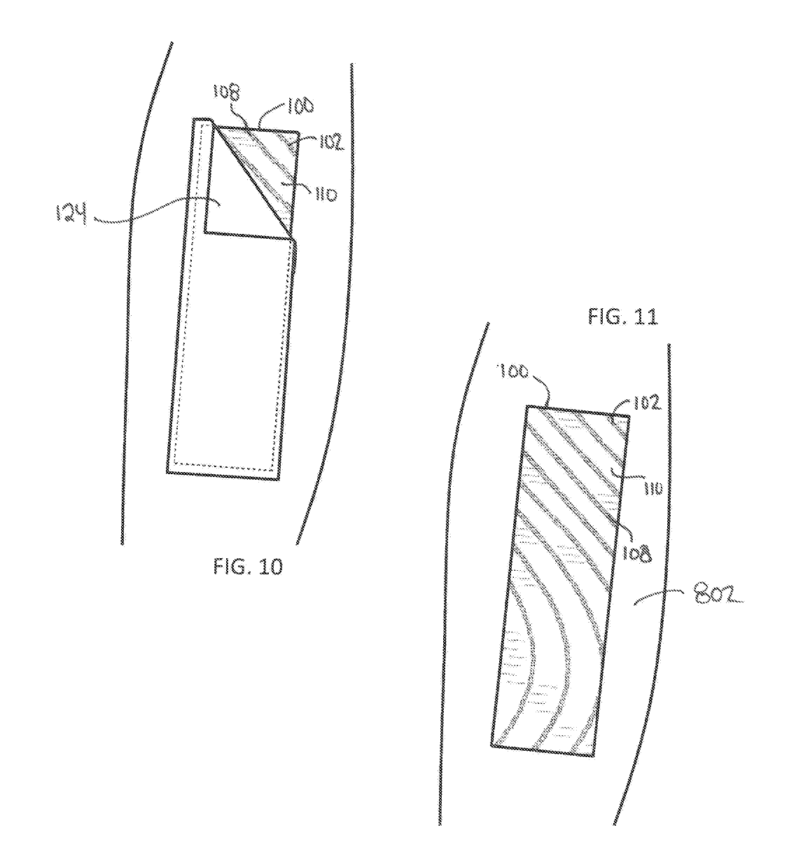

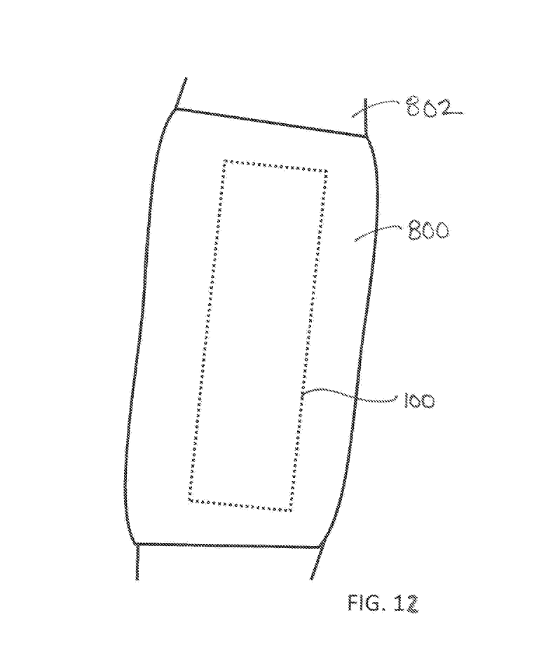

Referring to FIGS. 10 and 11, the second release liner 124 may then be removed from the tape 100 by peeling it from the second face 108 of the strip 102. Referring to FIG. 12, the shin pad 800 may then be adhered to the subject via the adhesive 110 on the second face 108. Pressure may then optionally be applied to the shin pad 800 (e.g. by pressing and rubbing the shin pad 800) to activate the adhesive 110. As mentioned above, further attachment mechanisms, such as straps, may be used in addition to the tape 100 to secure the shin pad 800 in place. For example, straps may be placed around the subject's leg to secure the shin pad to the leg, and the tape 100 may be used to prevent or inhibit the shin pad from rotating around the leg or shifting up or down on the leg while in use.

Due to the high stretch nature of the tape 100, as well as the discontinuous pattern of the adhesive 110, while in use the tape 100 may allow for and accommodate stretching and moving of the body, and allow for and accommodate slight movement of the shin pad 800.

Furthermore, as mentioned above, the tape may in some examples provide kinesiologic benefits to the subject. For example, the tape may support the body part to which it is adhered, in a similar manner to kinesiology tape. As such, the tape may be used both to attach objects to a body part and to support the body part.

In an alternative example, the tape 100 may be first be adhered to the object, and then be adhered to the skin.

In a further alternative example, several pieces of the tape 100 may be used to adhere the object to the skin. The pieces may optionally be spaced apart and positioned at different angles.

In any of the above examples, the tape 100 may be provided in a kit with instructions directing the user to carry out the method of FIGS. 8 to 12 (e.g. directing the user to remove the first release liner 122 and adhere the first face 106 to a subject's skin, and remove the second release liner 124 and adhere an object to the second face 108).

In any of the above examples, the adhesive may be applied on only a portion of the first face and/or second face. For example, in the tape 600, the adhesive may be applied on only the anchor portion 630 of the second face.

While the above description provides examples of one or more processes or apparatuses, it will be appreciated that other processes or apparatuses may be within the scope of the accompanying claims.

1. A tape for attaching objects to a subject comprising:
a) a strip of high stretch woven material having a first face and an opposed second face;
b) a first adhesive on the first face in a discontinuous pattern; and
c) a second adhesive on the second face.

2. The tape of claim 1, wherein the second adhesive is on the second face in a discontinuous pattern.

3. The tape of claim 2, wherein the first adhesive and the second adhesive overlay each other.

4. The tape of claim 2, wherein:
a) the first face includes a first set of spaced apart adhesive-covered portions that are covered by the adhesive, and a first set of spaced apart adhesive-free portions that are free of the adhesive; and
b) the second face includes a second set of spaced apart adhesive-covered portions that are covered by the adhesive, and a second set of spaced apart adhesive-free portions that are free of the adhesive.

5. The tape of claim 1, wherein the first adhesive and the second adhesive are a skin-safe adhesive.

6. The tape of claim 1, wherein the first adhesive and the second adhesive are a poly-acrylic adhesive.

7. The tape of claim 1, wherein the woven material is woven cotton.

8. The tape of claim 1, wherein the strip comprises an anchor portion and a plurality of fingers extending away from the anchor portion.

9. The tape of claim 1, wherein the strip has an unstretched length, and is elastically stretchable to a stretched length that is at least 1.4 times the unstretched length.

10. A kit for attaching objects to a subject comprising:
a) a tape comprising a strip of high stretch woven material having a first face and an opposed second face, a first adhesive on the first face in a discontinuous pattern, and a second adhesive on the second face;
b) a first release liner on the first face; and
c) a second release liner on the second face.

11. The kit of claim 10, wherein the tape, the first release liner, and the second release liner are provided in a roll.

12. The kit of claim 10, wherein the tape, the first release liner, and the second release liner are provided in individual precut pieces, wherein the tape of each individual precut piece comprises an anchor portion and a plurality of fingers extending away from the anchor portion.

13. The kit of claim 10, further comprising instructions directing a user to remove the first release liner and adhere the first face to a subject's skin, and remove the second release liner and adhere an object to the second face.

14. The kit of claim 10, wherein the second adhesive is on the second face in a discontinuous pattern.

15. The kit of claim 14, wherein:
a) the first face includes a first set of spaced apart adhesive-covered portions that are covered by the adhesive, and a first set of spaced apart adhesive-free portions that are free of the adhesive; and
b) the second face includes a second set of spaced apart adhesive-covered portions that are covered by the adhesive, and a second set of spaced apart adhesive-free portions that are free of the adhesive.

16. The kit of claim 10 wherein the first adhesive and the second adhesive are a skin-safe adhesive.

17. The kit of claim 10, wherein the first adhesive and the second adhesive are a poly-acrylic adhesive.

18. The kit of claim 10, wherein the woven material is woven cotton.

19. The kit of claim 10, wherein the strip comprises an anchor portion and a plurality of fingers extending away from the anchor portion.

20. The kit of claim 10, wherein the strip has an unstretched length, and is elastically stretchable to a stretched length that is at least 1.4 times the unstretched length.

21. A method for attaching an object to a subject, the method comprising:
a) removing a first release liner from a first face of a strip of high stretch woven material;
b) adhering the first face to the subject's skin via a first adhesive on the first face, the first adhesive in a discontinuous pattern on the first face;
c) removing a second release liner from a second face of the strip; and
d) adhering the object to the subject via a second adhesive on the second face.

22. The method of claim 21, wherein step b) comprises adhering the first face to the subject's skin while the strip is in an unstretched configuration.

23. The method of claim 21, wherein the object is body protective gear.

24. The method of claim 21, wherein the object is athletic gear.

25. The method of claim 21, wherein the second adhesive is in a discontinuous pattern on the second face.
